(19) United States
(12) Patent Application Publication
(10) Pub. No.: US 2004/0096527 A1
(43) Pub. Date: May 20, 2004

(54) Compositions for lowering serum cholesterol level

(76) Inventor: Jin-Hyuck Jang, Kumi-dong, Bundang-gu (KR)

Correspondence Address: BAKER & BOTTS, 30 ROCKEFELLER PLAZA, NEW YORK, NY, 10112

(21) Appl. No.: 10/469,086

(22) PCT Filed: Feb. 27, 2001
(86) PCT No.: PCT/KR01/00299

(51) Int. Cl. A61K 35/78

(52) U.S. Cl. 424/769; 424/773

(57) ABSTRACT

The present invention relates to compositions for lowering the serum cholesterol level, which contain (Yucca) extract and/or (Quillaja) extract. The compositions according to the invention, as the human-safe and medically functional compositions, can be used for various purposes comprising the prevention and treatment of cardiovascular disorders such as a hyperlipidemia, and intestinal disorders.

### TECHNICAL FIELD

The present invention relates to functional compositions comprising Yucca extract and Quillaja extract.

The present invention also relates to functional compositions for prevention and treatment of cardiac diseases and various diseases associated with gastro intestine which can lower serum cholesterol level upon taking the compositions, by using actions of various physiologically active substances including saponin existing in Yucca extract and Quillaja extract.

The compositions according to the present invention relates to functional compositions which can lower serum cholesterol level, particularly functional compositions which show lowering effect specific to low density cholesterol and neutral fat, and show prophylactic and therapeutic effects on gastro intestine-association diseases including constipation, diarrhea and digestion disorders.

The compositions according to the present invention is to provide an appropriate content ratio of Yucca extract and Quillaja extract contained in the compositions and optimal content ratio of other components so that the compositions can be taken as food and drink or for medical applications.

### BACKGROUND ART

Saponins, which are a group of glucoside compounds, are triterpenoid or steroid type, physiologically active, natural substances found in various plant groups and are known to have various beneficial effects such as surface activation, hematolysis, antimicrobial function, growth promotion, and complex formation with cholesterol, and the like. Therefore, saponins have been used as functional additives in various industrial fields such as feed, medicine, environment, and cosmetics, and at present are the subjects of many studies (Waller, G. R., Yamasaki, K., ‘Saponins used in Traditional and Modern Medicine’ 1996, Plenum Press).

Particularly, commercially available saponin fractions derived from Yucca schidigera and Quillaja saponaria extracts have been developed as aids of medicines including vaccine carrier, natural foaming agents and clean feed additives owing to their various functions (Korean Patent Application Nos. 1999-7002874, 1997-707527, 1996-046517, 1996-010630, 1997-0070702, 1997-0067052, 1991-0017141, etc.).

The Yucca schidigera extract and Quillaja saponaria extract are food additives derived from natural substances accepted by FDA (Food and Drug Administration) and are mostly used as natural foaming agents in products requiring stable foams. However, in this case, since the added amount is small, it is hard to expect various and beneficial physiological activities due to various components including saponins in the extracts.

Also, Yucca schidigera and Quillaja saponaria extracts are generally used separately. There are no products which use a combination of these extracts to attain synergy effects. Further, there is no functional composition for the prevention and treatment of cardiac diseases including lower serum cholesterol level and diseases associated with gastro intestine, which is derived from safe natural substances.

### DISCLOSURE OF INVENTION

It is, therefore, an object of the present invention to provide functional compositions containing Yucca schidigera extract and Quillaja saponaria extract. The compositions are functional compositions having serum cholesterol lowering effects and prophylactic and therapeutic effects on hyperlipidemia, various cardiac diseases, hypertension and diseases associated with gastro intestine. Among the compositions, compositions for drink are to develop functional compositions which are safe for human bodies and have an optimized flavor.

### BRIEF DESCRIPTION OF DRAWINGS

The above objects, features and advantages of the present invention will become more apparent from the following detailed description when taken in conjunction with the accompanying drawings, in which:

FIG. 1 is a graph showing changes in clinical conditions of a digest system before and after applying internally the functional composition VI according to an embodiment of the present invention, in which data is obtained by conducting questionnaires and scoring answers and mean values are shown;

FIG. 2 is a graph showing changes in serum cholesterol and neutral fat for entire clinical examination groups before and after applying internally the functional composition VI according to an embodiment of the present invention; and

FIG. 3 is a graph showing changes in serum cholesterol and neutral fat for the hyperlipidemia group having serum lipid level of 220 mg/dl before and after applying internally the functional composition VI according to an embodiment of the present invention.

### BEST MODE FOR CARRYING OUT THE INVENTION

Yucca schidigera is a plant belonging to the genus Yucca growing wild in the dry region of southwestern United states and its trunk and stems are used. A concentrate of Yucca juice prepared by mechanical pressing is called Yucca schidigera extract. Quillaja saponaria is a tree growing in the dry region of Chile in south America and its barks are mostly used due to their high saponin content. The Quillaja extract is prepared by boiling barks and lignum of Qyillaja and concentrating the resulting solution.

Components of the Yucca schidigera and Quillaja saponaria extracts can be separated into the butanol-extratable fraction and the non-butanol-extractable fraction. Saponins are contained in the butanol-extractable fraction. The non-butanol-extractable fraction contains carbohydrate type substances, showing characteristic physiological activities. Particularly, this fraction is known to promote the kidney function, affecting nitrogen metabolism in vivo, whereby the urea and ammonia level in blood can be lowered and pH of blood can be affected (R. J. Wallance et al., 1994, Appl. Environ. Microbiol., 60, 1762-1767).

The saponins contained in the butanol-extractable fraction are commercialized through separation from the representative two plants, i.e. Yucca schidigera and Quillaja saponaria. Two types of saponins from extracts of two plants show difference in their chemical structures, but basically comprise hydrophobic nucleus structures and hydrophilic side chains, by which they show various physiological activities. The nucleus structure of the Yucca saponin is represented by the following formula 1, which comprises a Yucca saponin nucleus of a steroidal structure and various side chains attached to a hydroxyl group of the Yucca saponin nucleus, producing various Yucca saponine groups.

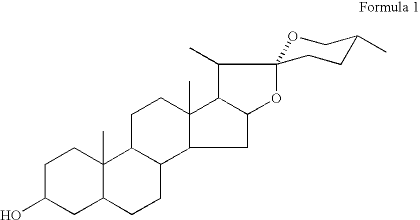

The entire structure of the Quillaja saponin is represented by the following formula 2, which is a macromolecule comprising a triterpenoidal nucleus portion and two carbohydrate side chains.

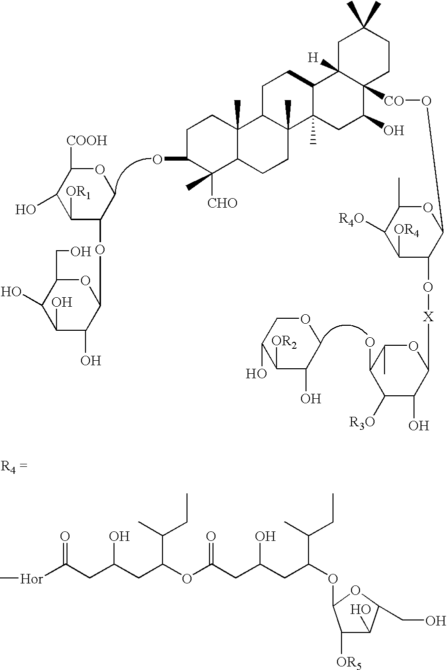

Yucca and Quillaja saponins are different in their numbers of side chains and charged radicals on the side chains, whereby they show difference in structure and physical property of the final micelles produced by binding with bile acid and cholesterol (Hostettmann, K., Marston, A., ‘Saponins’ 1995, Cambridge University Press).

Serum cholesterol is a main substance causing arteriosclerosis by attaching to blood vessel together with salts and thereby, interfering with blood flow. Cholesterol and lipid in vivo are synthesized by taken fat.

Fat metabolism in vivo is performed as follows. Fats primarily digested after ingestion are mostly long-chain neutral fats (long-chain triglycerides) and are mixed with gastric juice in the stomach, followed by entrance into the duodenum. The neutral fats and the gastric juice reaching the duodenum stimulate secretin and chloecystokinin, upon which the bile and pancreatin are secreted. Then, the neutral fats are separated into fatty acids and monoglycerides by the pancreatin and the fatty acids and monoglycerides come into the bile which takes in the form of micelle in the bowel to form mixed micelles. The mixed micelles absorb only the fatty acids and monoglycerides through hydrophilic intestinal mucosa membrane. Here, 90% of the bile which is not absorbed is absorbed into ileum to be reused and the rest is evacuated in the excrement. The fatty acids and monoglycerieds absorbed as above are used in lipid and cholesterol synthesis in the liver and the loss of the bile is replenished through cholesterol metabolism in the liver (Norton J. Greenberg, Kurt J. Isselbacher, 1998, Harrison's Principles of Internal Medicine, McGraw-Hill Press, 14^{th} edition, 1616-1633).

The saponins participate in cholesterol synthesis and metabolism in vivo due to their chemical structures similar to that of cholesterol. In the gastrointestinal tract, the saponins directly bind to and capture cholesterol, thereby interfering with absorption of cholesterol. Also, the saponins strongly bind to bile-mixed micelles to form insoluble complexes, which are then excreted through the colon. Therefore, they can inhibit absorption of cholesterol in the small intestines and prevent oxidation of cholesterol in the colon (Tagaki S., Otskura, H., Akyama, T., Sankawa, U., 1982, Chem. Pharm. Bull., 30, 3485-3492). In addition, the saponins directly react with the bile acid to form polymers, which are then excreted, whereby they can inhibit formation of micelles between the fat lysates and the bile, hindering the absorption of fatty acids and monoglycerides in vivo through the intestinal mucosa (Gee, J. M., Johnson, I. T., 1988, J. Nutr., 118, 1391-1397). Thus, when the bile concentration is reduced through various routes due to saponins, the cholesterol metabolism is promoted on the bile synthesis route for supplement, whereby the serum cholesterol level is reduced. In addition, the saponins affect the strengthening of immunity by promotion of permeability and absorption of antimicrobial arthritis treating agent specific to protozoans and gram-positive bacteria in the internal organs due to their surface activities.

Hyperlipidemia is a risk factor developing cardiovascular diseases and is associated with angina pectoris and mycocardial infarction. According to studies including Multiple Risk Factor Intervention Trial (MRFIT), it is shown that as serum cholesterol level increases 1%, the risk of coronary artery diseases increases 2%. Lipid in the body is classified into cholesterol and neutral fats. The cholesterol is largely divided into a high density cholesterol and a low density cholesterol. It is known that the high density cholesterol is an anti-arteriosclerosis factor and increase of the low density cholesterol level is a most important cause of cardiovascular diseases. That is, when the high density cholesterol level is high and the low density cholesterol level is low, the cardiovascular diseases can be prevented. On the contrary, when the high density cholesterol level is low and the low density cholesterol level is high, the cardiovascular disease is induced. The neutral fats are also known as important risk factors. National Cholesterol Education Program (NCEP) published guidelines for managing fat level in blood in 1993, as shown in Table 1.

TABLE 1

| | Total | Low density | | High density |
|---|---|---|---|---|
| | cholesterol | cholesterol | Neutral fats | cholesterol |
| Ideal value | <200 | <130 | <200 | >60 |
| Warning value | 200-239 | 130-159 | 200-400 | |
| Value needing | >240 | >160 | >400 | <35 |
| treatment | | | | |

Therefore, the control of the low density cholesterol in blood is very important in a primary or secondary prevention of cardiovascular diseases. Subjects of primary prevention treatment, without cardiovascular disease, include cases having other risk factors of less than 2 and a low density cholesterol level in blood of 160 mg/dl or more and cases having other risk factors of 2 or more and a low density cholesterol level in blood of 130 mgidl or more. In case of subjects with cardiovascular disease of secondary prevention treatment, the low cholesterol level in blood should be maintained 100 mg/dl or less. Here, the primary prevention is to prevent patients who have not had any symptoms of disease from the disease and the secondary prevention is to perform treatment of patients who have shown any symptoms of disease so as to intercept the progress of the disease.

As described above, the low density cholesterol level is a critical factor to determine possibility of development of cardiovascular disease. For the ideal treatment of hyperlipidemia, the high density cholesterol level does not decrease but only the low density cholesterol level should decrease. When the low density cholesterol level does not decrease although the cholesterol level decreased, prophylactic effect on cardiovascular diseases prevention cannot be expected (Joseph L. Witzum, Daniel Steinberg, 2000, Cecil Testbook of Medicine, 21^{st} Edition, Sauders, 1090-1100).

The functional compositions having the serum cholesterol level lowering effect and containing Yucca extract and Quillaja extract according to the present invention was confirmed to effectively reduce total cholesterol in blood in clinical examinations. Particularly, the compositions lower the lipid concentration in blood, specifically to the low density cholesterol and neutral fats which are harmful to human bodies while they do not significantly affect the high density cholesterol level which are useful for human bodies. Accordingly, the functional compositions are expected to show superior prophylactic and therapeutic effects on cardiovascular diseases. Also, the functions compositions according to the present invention showed eye-opening effects in solution of various disorders associated with gastro intestine such as constipation, diarrhea, feeling full and abdominal distention, hangover, etc.

Therefore, the functional compositions having the serum cholesterol level lowering effect and containing Yucca extract and Quillaja extract according to the present invention can be used in prevention and treatment of various cardiovascular disease and hypertension as well as hyperlipidemia, and show prophylactic and therapeutic effects on disorders associated with gastro intestine. Also, the compositions are safe to human bodies.

### EXAMPLE

Functional compositions containing Yucca and Quillaja extracts and carbonated, non-carbonated and diluted drink compositions were prepared.

For the carbonated drinks, based on the weight of solid contents, 0 to 5 wt % of Yucca extract and 0 to 3 wt % of Quillaja extract, preferably 0.01 to 1.0 wt % of Yucca extract and 0.005 to 1.0 wt % of Quillaja extract, more preferably 0.01 to 0.3 wt % of Yucca extract and 0.005 to 0.2 wt % of Quillaja extract were added to conventional ingredients of cola and cider.

For the non-carbonated drinks, based on the weight of solid contents, 0 to 5 wt % of Yucca extract and 0 to 3 wt % of Quillaja extract, preferably 0.01 to 1.0 wt % of Yucca extract and 0.005 to 1.0 wt % of Quillaja extract, more preferably 0.01 to 0.3 wt % of Yucca extract and 0.005 to 0.2 wt % of Quillaja extract were added to conventional ingredients of vegetable and fruit juice.

### Example 1

### Carbonated Drink Composition I

Yucca extract 0.2%, Quillaja extract 0.01%, liquid fructose 14.5%, caramel 0.12%, citric acid 0.1%, caffeine 0.009%, carbonic acid gas 0.77%, flavor 0.06%, purified water for the balance

### Example 2

### Carbonated Drink Composition II

Yucca extract 0.15%, Quillaja extract 0.015%, sugar 7.5%, citric acid 0.05%, Vitamin C 0.05%, coloring agent 0.001%, flavor 0.1%, amino acid 0.003%, a synthetic sweetener 0.06%, purified water for the balance

### Example 3

### Non-Carbonated Drink Composition III

Yucca extract 0.25%, Quillaja extract 0.01%, liquid fructose 5.15%, orange concentrate (65 brix) 13.85%, Vitamin C 0.05%, citric acid 0.06%, flavor 0.1%, purified water for the balance

### Example 4

### Non-Carbonated Drink Composition IV

Yucca extract 0.2%, Quillaja extract 0.02%, sugar 7.2%, citric acid 0.12%, a synthetic sweetner 0.008%, Vitamin C 0.04%, natural pigment 0.05%, flavor 0.08%, purified water for the balance

### Example 5

### Diluted Drink Composition V

Yucca extract 1.0%, Quillaja extract 0.04%, liquid fructose 58.18%, caramel 0.52%, citric acid 0.32%, Vitamin C 0.2%, taurine 0.814%, purified water for the balance

### Example 6

### Diluted Drink Composition VI

Yucca extract 57.0%, Quillaja extract 30.0%, Vitamin A 0.15%, Vitamin D 0.2%, Vitamin B2 0.15%, Vitamin C 0.2%, taurine 3.0%, polydextrose 2.5%, purified water for the balance

### Example 7

### Clinical Examination, Measurement of Lipid in Blood and Questionnaire Survey

56 Healthy females, with a average age of 56.9±6.6 (minimum 40 and maximum 77), were finally selected from the subject group by excluding persons who had taken drugs affecting hyperlipidemia within 2 months and had cardiovascular diseases.

Prior to the experiment, members of the subject group were measured for their lipid level in blood and classified into the hyperlipidemia group having a serum cholesterol level of 220 mg/dl or more and the normal group having a serum cholesterol level of less than 220 mg/dl. The members of the hyperlipidemia group were 27 personnel, with average age of 57.7±7.5 (minimum 40, maximum 77).

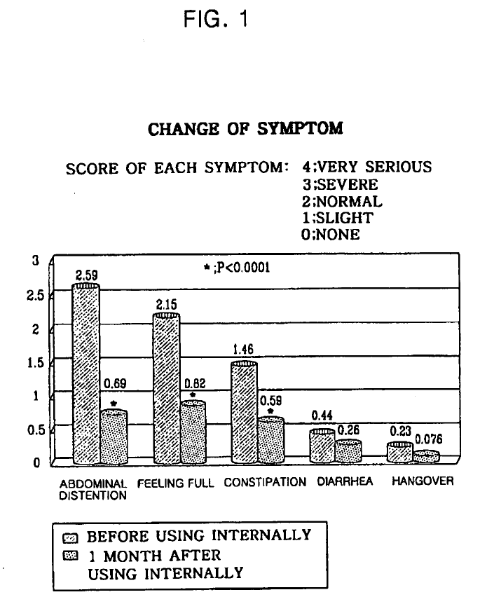

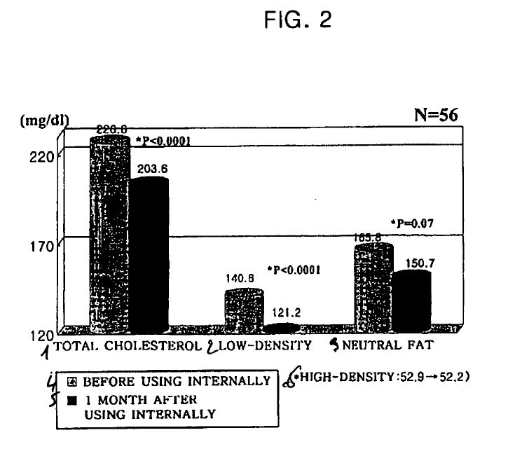

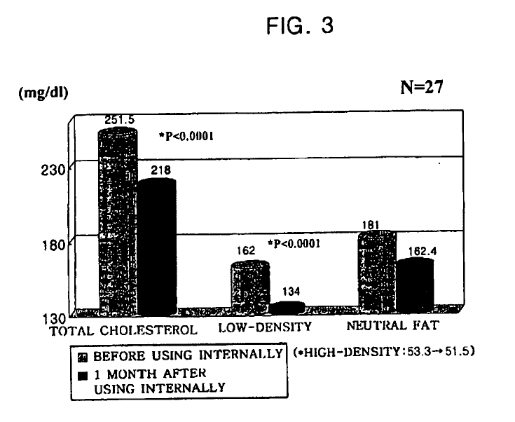

For one month, the subjects daily took 0.9 cc of the functional composition VI of Example 6 using a 1 cc syringe and mixed 0.33 of the composition with 150 or 200 cc of drink (normal drinking water). They use internally the mixture three times per day. Before and after using internally the composition, the subjects answered the questionnaire, followed by scoring (FIG. 1) and after abstinence, were subjected to the measurement of lipid in blood (FIG. 2: the entire clinical examination group; FIG. 3: hyperlipidemia group).

The measurement of lipid in blood was performed before and one month after using internally the composition. For the measurement, the subjects were fasted from 10 pm of previous day and maintain an empty stomach for at least 10 hours before blood collecting. The measurement was performed using Vitros 750 model, produced by Johnson & Johnson Co. (USA).

In order to demonstrate the effects on disorders associated with gastro intestine, questionnaire survey was conducted for subjects before and after using internally the composition of Example 6. The subjects replied the questionnaire as 5 grades (0: none; 1: slight; 2: normal; 3: slightly severe; 4: very severe) in terms of 5 symptoms (constipation, diarrhea, digestion disorders, abdominal distension, and hangover) with respect to the difference between before and after internally using the composition.

The data was analyzed according to the paired t-test method using SPSS statistic program. When p value is less than 0.05, the statistic difference is significant and as the p vale gets smaller, the valence increased.

As shown in FIG. 1, the compositions according to the present invention had eye-opening effects on various disorders associated with gastro intestine such as constipation, diarrhea, feeling full and abdominal distention, hangover, etc. Also, the composition effectively lowered the low density cholesterol level and neutral fat level without affecting the high density cholesterol level in blood after 4 weeks application, which confirmed effectiveness of the composition in prevention and treatment of various cardiovascular diseases including hyperlipidemia (FIG. 2 and FIG. 3). Numerically, the entire subjects showed significantly lowered total cholesterol level of 10% (P<0.0001), low density cholesterol level 13% (P<0.001) and neutral fat level of 11% (P=0.07). Particularly, in case of hyperlipidemia having the initial total cholesterol level of 220 mg/dl or more, the subjects showed 14% reduced cholesterol level (P<0.0001), 17% reduced low density cholesterol level (P<0.0001) and 13% reduced neutral fat level (no valence). Therefore, it was noted that the functional compositions according to the present invention are more effective on hyperlipidemia.

### INDUSTRIAL APPLICABILITY

According to the present invention, functional compositions having the serum cholesterol level lowering effect and containing Yucca extract and Quillaja extract according to the present invention are provided.

Through clinical examinations, it was confirmed that the compositions have the serum cholesterol level lower effects and it was suggested that the compositions can be applied in functional food and drinks having prophylactic and therapeutic effects on hyperlipidemia. Particularly, the compositions according to the present invention lower the lipid concentration in blood, specifically to the low density cholesterol and neutral fats which are harmful to human bodies while they do not significantly affect the high density cholesterol level which are useful for human bodies. Accordingly, the functional compositions are expected to show superior prophylactic and therapeutic effects on cardiovascular diseases. Thus, the functional compositions having the serum cholesterol level lowering effect and containing Yucca extract and Quillaja extract according to the present invention will be applicable in prevention and treatment of various cardiac diseases and hypertension as well as hyperlipidemia.

Also, the functions compositions according to the present invention showed eye-opening effects in solution of various disorders associated with gastro intestine such as constipation, diarrhea, feeling full and abdominal distention, hangover, etc. Therefore, they can be infinitely used in safe functional compositions which are safe to human bodies and have prophylactic and therapeutic effects on disorders associated with gastro intestine.

The compositional ratios described in respective examples and claims of the present invention are determined for optimal taste as drinks and for physiological activities as functional compositions and can be applied in the industrial field.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A composition having a serum cholesterol level lowering effect and therapeutic and prophylactic effects of cardiovascular diseases, comprising at least 0.001 wt % of Yucca extract and at least 0.001 wt % of Quillaja extract, based on solid contents.

2. A composition having therapeutic and prophylactic effects of disorders associated with gastro intestine, comprising at least 0.001 wt % of Yucca extract and at least 0.001 wt % of Quillaja extract, based on solid contents.

3. The composition of claim 1, wherein the composition lowers specifically a low density cholesterol level and a neutral fat.

4. The composition of claim 1 or 2, wherein the composition is used as foods and drinks.

5. The composition of claim 1 or 2, wherein the composition is for medicinal applications.

6. The composition of claim 1 or 2, wherein the Yucca extract and Quillaja extract in a liquid phase are used in an amount of 0.1 to 70% by weight of evaporated solid contents, or the Yucca extract and Quillaja extract in solid phase are used in a extract concentration of 0.01 to 100 wt %.

7. The composition of claim 4, wherein the drinks are selected from carbonated drinks, agricultural product drinks, natural food drinks, and diluted drinks.

8. The composition of claim 7, wherein the carbonated drinks contain 0 to 5 wt % of Yucca extract and 0 to 3 wt % of Quillaja extract, based on the solid contents.

9. The composition of claim 8, wherein the carbonated drinks contain 0.01 to 1.0 wt % of Yucca extract and 0.005 to 1.0 wt % of Quillaja extract, based on the solid contents.

10. The composition of claim 9, wherein the carbonated drinks contain 0.01 to 0.3 wt % of Yucca extract and 0.005 to 0.2 wt % of Quillaja extract, based on the solid contents.

11. The composition of claim 7, wherein the agricultural product drinks and natural food drinks contain 0 to 5 wt % of Yucca extract and 0 to 3 wt % of Quillaja extract, based on the solid contents.

12. The composition of claim 11, wherein the agricultural product drinks and natural food drinks contain 0.01 to 1.0 wt % of Yucca extract and 0.005 to 1.0 wt % of Quillaja extract, based on the solid contents.

13. The composition of claim 12, wherein the agricultural product drinks and natural food drinks contain 0.01 to 0.3 wt % of Yucca extract and 0.005 to 0.2 wt % of Quillaja extract, based on the solid contents.

14. The composition of claim 1 or 2, wherein the content ratio of Yucca extract to Quillaja extract is 0:100 to 100:0, more preferably 10:90 to 90:10.

15. The composition of claim 14, wherein the content ratio of Yucca extract to Quillaja extract is 10:90 to 90:10.
